(19) United States
(12) Patent Application Publication
(10) Pub. No.: US 2025/0241662 A1
(43) Pub. Date: Jul. 31, 2025

(54) GUIDEWIRE-CAPTURING SURGICAL INSTRUMENT

(71) Applicant: GLOBUS MEDICAL, INC., AUDUBON, PA (US)

(72) Inventors: Caelan Allen, Ambler, PA (US); George Yacoub, Lansdale, PA (US); Matthew Bechtel, Philadelphia, PA (US)

(21) Appl. No.: 18/422,051

(22) Filed: Jan. 25, 2024

(51) Int. Cl. A61B 17/16; A61B 17/00

(52) U.S. Cl. CPC A61B 17/1697; A61B 17/1613; A61B 2017/00367; A61B 2017/00982

(57) ABSTRACT

A guidewire capturing device may include a handle that may have a central aperture extending in an axial direction therethrough. The device may also include a wire housing having a wire aperture extending in the axial direction therethrough, the wire aperture may have a guidewire positioned therewithin. The wire housing may be at least partially contained within the central aperture and selectively movable in the axial direction within the central aperture from a first position to a second position. The device may include a clutch, which may be connected to the handle and selectively movable between an engaged position and a disengaged position, the engaged position corresponding to a threaded engagement between the wire housing and the clutch, and the disengaged position corresponding to free axial movement of the wire housing within the central aperture between the first position and the second position.

### TECHNICAL FIELD

This disclosure relates to a surgical instrument that may be used to control a guidewire and insert a pedicle screw.

### BACKGROUND

In various orthopedic surgeries, various instruments may be used to implant hardware into a patient. Having instruments that are easier to use or require fewer steps may improve the surgical process, reduce cost, and improve patient outcomes. Accordingly, what is needed are systems and methods for surgical instruments that are easier to use and require fewer steps.

### SUMMARY

Disclosed herein are implementations of a guidewire-capturing instrument The guidewire-capturing instrument may comprise a handle comprising a central aperture extending in an axial direction therethrough, wherein the central aperture may be centered about a central axis extending in the axial direction. The guidewire-capturing instrument may also include a wire housing having a wire aperture extending in the axial direction therethrough. The wire aperture may be sized and shaped to receive a guidewire and may be at least partially contained within the central aperture and selectively movable in the axial direction within the central aperture from a first position to a second position. The guidewire-capturing instrument may also include a clutch connected to the handle that may be selectively movable between an engaged position and a disengaged position, the engaged position corresponding to a threaded engagement between the wire housing and the clutch, and the disengaged position corresponding to free axial movement of the wire housing within the central aperture between the first position and the second position.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIGS. 1A and 1B are perspective views of examples of a guidewire-capturing instrument.

FIG. 2A is a side view of an example of the guidewire-capturing instrument shown in FIGS. 1A and 1B.

FIG. 2B is an exploded side view of an example of a guidewire-capturing instrument shown in FIGS. 1A and 1B.

FIG. 2C is an exploded side view of an example of the guidewire-capturing instrument of FIG. 2A with the clutch in the engaged position.

FIG. 2D is an exploded side view of an example of the guidewire-capturing instrument of FIG. 2A with the clutch in the disengaged position.

FIG. 3 is an exploded perspective view of an example of the wire housing of FIG. 2A.

FIG. 4 is a perspective view of an example of the guidewire and wire housing within the guidewire-capturing instrument.

FIG. 5A is a cross-sectional view of an example of the driver housing with the driver sleeve in the first state.

FIG. 5B is a cross-sectional view of an example of the driver housing with the driver sleeve in the second state.

FIG. 5C is an exploded side view of an example of the driver housing.

FIG. 6A is a cross-sectional view of an example of the guidewire-capturing instrument with a guidewire inserted.

FIG. 6B is a cross-sectional view of an example of the wire housing with a guidewire inserted with the clutch engaged.

FIG. 6C is a cross-sectional view of an example of the wire housing with a guidewire inserted with the clutch disengaged.

FIG. 7 is a flow diagram of an example of a first method of use for the guidewire-capturing instrument with a driver, guidewire, and a screw.

FIG. 8 is a flow diagram of an example of a second method of use for the guidewire-capturing instrument with a driver, guidewire, and a screw.

FIG. 9 is a flow diagram of an example of a third method of use for the guidewire-capturing instrument with a driver, guidewire, and a screw.

### DETAILED DESCRIPTION

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law. Note that in the drawings, dimensions, ratios, and the like may not be illustrated to completely coincide with actual ones. In addition, details may be omitted depending on the drawings.

The conventional procedure for percutaneous pedicle screw insertion under fluoroscopy involves the insertion of a bone access needle into a bone, followed by the insertion of a guide wire into the bone. A pedicle screw may be inserted over the guide wire, which may keep the pedicle screw on a prescribed trajectory established by a surgeon. The conventional procedure requires a minimum of three separate instruments to insert a pedicle screw. Using multiple instruments for pedicle screw insertion increases the risk of infection for the patient and complicates the procedure, as each instrument must be properly sterilized, and the surgeon must switch instruments multiple times during the procedure. It would prove advantageous to utilize a system that can perform each of these steps with a single instrument. Such an instrument may reduce surgical time and the risk of infection. Additionally, such an instrument may also reduce the risk of accidental breach into non-targeted tissue (i.e. the vertebral foramen or body) and reduce guidewire kinking or fracture.

A system of surgical instruments, implants, and methods may be used to reduce the number of surgical steps to implant a screw into tissue through a minimally sized incision. A screw, among other fixators and complementary implants may be used to fixate tissue and facilitate healing. The components of such a system may include an instrument that can capture, release, advance, and retract a guidewire or stylet, as well as a screw (such as a pedicle screw) with an embedded guidewire. The system may work in conjunction with other instruments to implant a full screw construct via a minimal incision. Similarly, the system may be adapted for specific non-surgical or surgical uses. For example, the system may be used to implant screw constructs in the thoracolumbar and sacral spine region for primary fixation and may treat a variety of conditions including degenerative disc disease, spondylolisthesis, trauma (i.e., trauma or dislocation), and/or tumors.

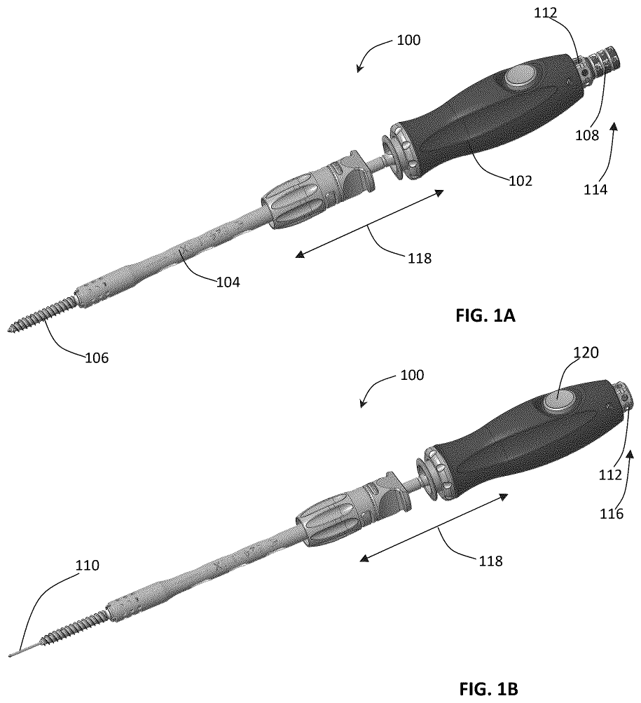

FIGS. 1A and 1B are diagrams of examples of a guidewire-capturing instrument 100. FIG. 1A includes the guidewire-capturing instrument 100, a handle 102, a screwdriver bit 104, a screw 106, a wire housing 108, a collar 112, a first position 114, and an axial direction 118. FIG. 1B includes the guidewire-capturing instrument 100, a guidewire 110, a clutch 120, and a second position 116. FIG. 1B also includes the axial direction 118, and the collar 112 shown in FIG. 1A.

The guidewire-capturing instrument 100 may be part of a single-step system for pedicle screw insertion. The guidewire-capturing instrument 100 may be used in conjunction with the screw 106 (e.g., a pedicle screw) shown in FIG. 1A and the guidewire 110 shown in FIG. 1B. Referring to FIG. 1A, the handle 102 may at least partially house the wire housing 108, and the wire housing 108 may at least partially contain the guidewire 110. The handle 102 may detachably connect to the screwdriver bit 104, and the screwdriver bit 104 may detachably connect to the screw 106.

The handle 102 may function to actuate the guidewire 110 shown in FIG. 1B for a fixed distance with respect to the screw 106 shown in FIG. 1A. The handle 102 may also function to implant or remove the screw 106 from a bone. The screwdriver bit 104 may function to connect with the screw 106, or a variety of other orthopedic screws. The screwdriver bit 104 may provide stability for an orthopedic screw during implantation into or removal from a bone. The screwdriver bit 104 may also translate rotational force from the handle 102 to the screw 106. The screw 106 may function as a tissue fixator and as a channel through which the guidewire 110 may pass while providing stability for the guidewire 110 during implantation or removal.

The wire housing 108 may function to detachably hold an end of the guidewire 110. The guidewire 110 may function to create a trajectory within bone for the screw 106. The guidewire 110 may be advanced or retracted a fixed distance through the screw 106.

A wire housing 108 may move within a handle 102 through a defined range of motion. A collar 112 may be positioned around the wire housing 108 and define the range of motion. The range of motion of the wire housing 108 may extend from a first position 114 to a second position 116. In the first position 114, the wire housing 108 may extend in an axial direction 118 out of the handle 102. The first position 114 may correspond to the guidewire 110 being retracted (e.g. maximally retracted) within the screw 106. In the second position 116, the wire housing may be retracted in an axial direction 118 within the handle 102. The second position 116 may correspond to the guidewire 110 being extended (e.g. maximally extended) out of the screw 106. The wire housing 108 may be fully extended out of the handle 102 and the collar 112 in the first position 114, and the wire housing 108 may be fully retracted into the handle 102 and the collar 112 in the second position 116. However, these are merely examples of the first position 114 and the second position 116.

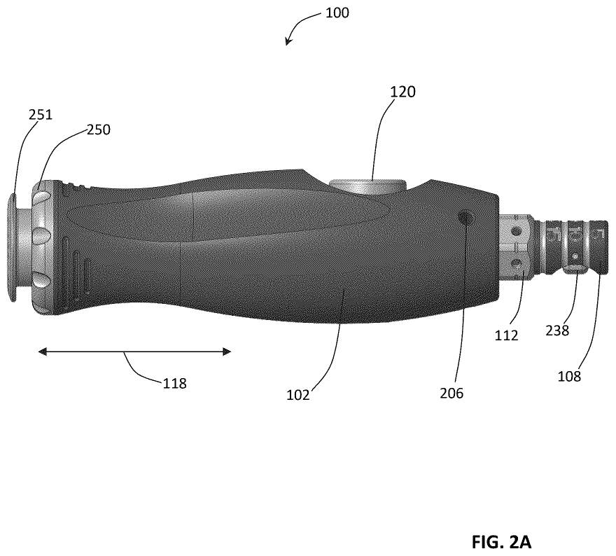

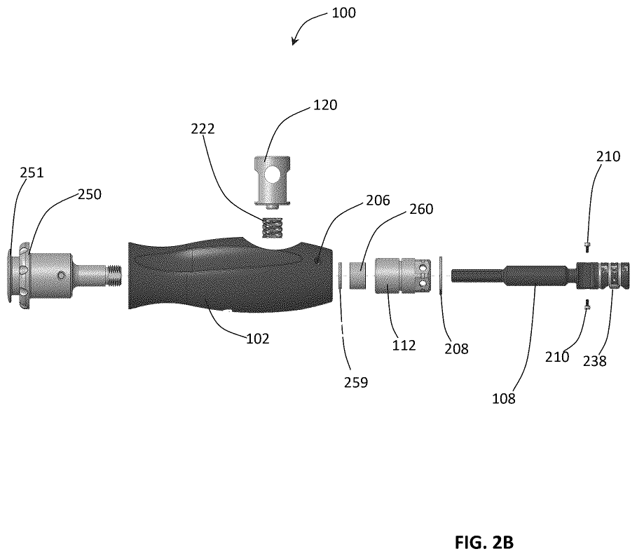

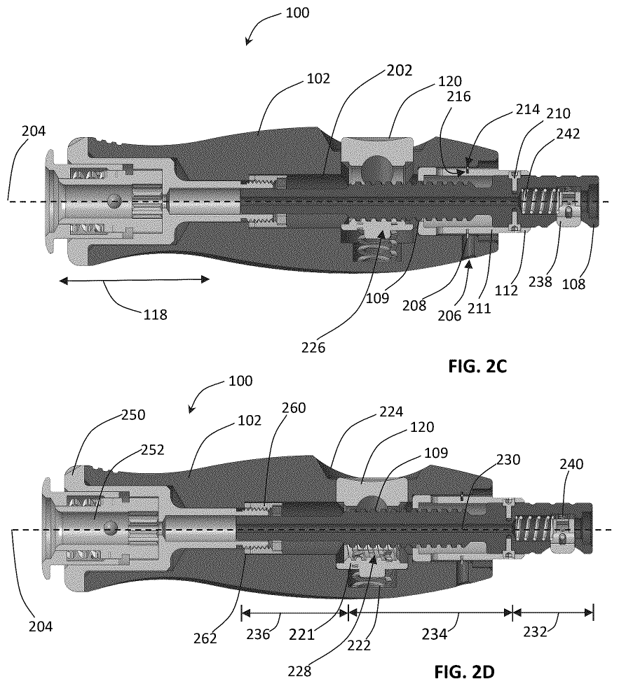

FIG. 2A is a side view of an example of the guidewire-capturing instrument 100 shown in FIGS. 1A and 1B. FIG. 2A includes the handle 102, the wire housing 108, the collar 112, the clutch 120, and the axial direction 118 shown in FIGS. 1A and 1B. FIG. 2A also includes an access aperture 206, a lock 238, a driver housing 250, and a driver sleeve 251.

FIG. 2B is an exploded side view of an example of the guidewire-capturing instrument 100 shown in FIGS. 1A and 1B. FIG. 2B includes the handle 102, the wire housing 108, the collar 112, the clutch 120, the access aperture 206, the lock 238, the driver housing 250, and the driver sleeve 251 of FIGS. 1A-2A. FIG. 2B also includes a lock ring 208, collar screws 210, a clutch spring 222, a retention ring 259, and a nut 260.

FIG. 2C is an exploded side view of an example of the guidewire-capturing instrument 100 of FIG. 2A with the clutch in the engaged position. FIG. 2C includes the handle 102, the wire housing 108, wire housing threads 109, the collar 112, the axial direction 118, the clutch 120, the access aperture 206, the lock ring 208, the collar screws 210, and the lock 238 of FIGS. 1A-2B. FIG. 2C also includes a central aperture 202, a central axis 204, a collar slot 211, a first groove 214, a second groove 216, an engaged position 226, and an aperture spring 242.

FIG. 2D is an exploded side view of an example of the guidewire-capturing instrument 100 of FIG. 2A with the clutch in the disengaged position. FIG. 2D includes the handle 102, the wire housing threads 109, the central axis 204, the clutch spring 222, the driver housing 250, and the nut 260 of FIG. 2C. FIG. 2D also includes clutch threads 221, a handle recess 224, a disengaged position 228, a wire aperture 230, a first portion 232, a second portion 234, a third portion 236, a lock spring 240, a driver aperture 252, and a washer 262.

Referring to FIGS. 2A-2D, the handle 102 may be sized and shaped so that a human user may grasp it with one hand during a surgical procedure. For example, the handle 102 may have an elongated or generally cylindrical shape so that it may fit within the closed grasp of a user. The handle 102 may be shaped in a variety of ergonomic shapes. The handle 102 may also be sized and shaped to be grasped by a robotic surgical device.

Referring to FIG. 2C, the handle 102 may have a central aperture 202 extending in an axial direction 118 therethrough. The central aperture 202 may extend for the entire length of the handle. Furthermore, the central aperture 202 may be centered about a central axis 204. The central aperture 202 may be shaped so that it is symmetrical about the central axis 204. Alternatively, the central aperture 202 may be shaped so that is asymmetrical about the central axis 204. For example, the central aperture 202 may be wider or narrower at some points along the direction of the central axis 204. The central aperture 202 may also include features (e.g. notches, and cut-outs) that are not symmetrically reflected about the central axis 204. The central aperture 202 may also contain at least a portion of the wire housing 108. The central aperture 202 may function as a host for at least part of additional components and allow for modular customizability of the guidewire-capturing instrument 100. The central aperture 202 may also allow for complete deconstruction or construction of the guidewire-capturing instrument 100 by granting a user access to each of the components of the guidewire-capturing instrument 100.

FIGS. 2A-2C show the collar 112 of the guidewire-capturing instrument 100. The collar 112 may be at least partially contained by the central aperture 202 and extend circumferentially around at least a portion of the wire housing 108. The collar 112 may be centered about the central axis 204. The collar 112 may include a slot 211 that interacts with a collar screw 210 extending into the wire housing 108. The collar 112 may function as an environment providing the bounds for axial movement of the wire housing 108. The dimensions of the collar 112 may define a first position and second position between which the wire housing 108 may move.

The collar 112 may also be generally cylindrical and may have an outer hexagonal surface. The outer surface of the collar 112 may have flat sides or also be polygonal. The outer surface of the collar 112 may have edges that do not interfere with interior walls of the handle 102 as defined by the central aperture 202. The outer surface of the collar 112 may function to mate with a wrench, a driver handle, or some other tool to impart rotation. The collar 112 may also function to translate rotation from an exterior source to wire housing 108 about central axis 204.

FIGS. 2B and 2C show the lock ring 208 of the guidewire-capturing instrument 100. The lock ring 208 may secure the collar 112 and prevent the collar 112 from unrestricted axial motion within central aperture 202. For example, when in place, the lock ring 208 may simultaneously engage a first groove 214 formed in the handle 102 and a second groove 216 formed in the collar 112 to secure the collar 112 within the central aperture 202.

Referring to FIGS. 2A and 2C, the handle 102 of the guidewire-capturing instrument 100 may include the access aperture 206. The access aperture 206 may extend from an exterior surface of the handle 102 and into the central aperture 202. The orientation of the access aperture 206 may be orthogonal to that of the central aperture 202. Alternatively, it may be that the orientation of the access aperture 206 is not orthogonal to that of the central aperture. The location of the access aperture 206 on the handle 102 may correspond to the location of the collar 112 within the central aperture. The access aperture 206 may function to allow access to the collar 112 and to the lock ring 208. For example, to remove the collar 112 from the handle 102 a user may depress the lock ring 208 via the access aperture 206, allowing the user to slide the collar 112 out of the handle 102.

Referring to FIGS. 2B and 2C, the guidewire-capturing instrument 100 may include one or more collar screws 210. One or more collar screws 210 may be used in tandem with the collar 112 and the wire housing 108. A collar screw 210 may extend (i.e. be embedded fixedly or removably) into at least a part of the wire housing 108, and the head of the collar screw 210 may protrude out of the wire housing 108 and into a slot 211 in the collar 112. The collar screw 210 may maintain the positioning of the wire housing 108 and its relative motion with respect to collar 112. For example, the collar screw 210 may abut against a slot 211 or space in the collar 112 to prevent rotational motion of wire housing 108 and define the limits of axial motion of the wire housing 108 within the collar 112.

FIGS. 2A-2C show the clutch 120 of the guidewire capturing instrument 100. The clutch 120 may control how the wire housing 108 moves within the central aperture 202. For example, when the clutch 120 is in an engaged position 226, the clutch 120 engages the wire housing 108 and controls (e.g. restricts) movement of the wire housing 108 in the axial direction 118. When the clutch 120 is in a disengaged position 228, the clutch 120 releases the wire housing 108 and frees the wire housing 108 to move in the axial direction 118.

The clutch 120 may be configured in any of a variety of forms including but not limited to levers, knobs, dials, switches, or other actuators. For example, the clutch 120 may be in the form of a button that protrudes from the handle 102 and extends at least partially into the central aperture 202. The clutch 120 may be biased by a clutch spring 222 or some other biasing mechanism. For example, the clutch spring 222 may be a coil spring. The clutch spring 222 may effectively spring load the clutch 120 so that it must be actively depressed by a user to toggle or otherwise move between its settings.

For example, the clutch spring 222 may bias or press against the bottom (i.e. the distal portion) of the clutch 120 so that the default position of the clutch 120 is in the engaged position 226. This may require a user to actively depress the clutch 120 to move it into the disengaged position 228 and when the user releases the clutch 120, the clutch spring 222 may act to return the clutch 120 to the engaged position 226. Alternatively, the clutch spring 222 may bias or press against the bottom of the clutch 120 so that the default position of the clutch 120 is in the disengaged position 228. This may require a user to actively depress the clutch 120 to move it into the engaged position 226 and when the user releases the clutch 120, the clutch spring 222 may act to return the clutch 120 to the disengaged position 228.

The clutch 120 may restrict axial movement of the wire housing 108 in the axial direction 118. For example, the clutch 120 may include one or more clutch threads 221 on the inside surface of the clutch 120. The clutch threads 221 may complementarily engage with the wire housing threads 109. The clutch threads 221 may releasably engage with the wire housing threads 109 when the clutch 120 is in the engaged position 226. The clutch threads 221 may disengage with the wire housing threads 109 when the clutch 120 is in the disengaged position 228.

The clutch threads 221 may have a pitch that is complementary to the pitch of one or more wire housing threads 109. This may allow for the clutch threads 221 and the wire housing threads 109 to smoothly engage and slide past one another in relative rotation (e.g., when the wire housing 108 is rotated about the central axis 204 with respect to the clutch 120 and/or the handle 102). For example, when the clutch 120 is in the engaged position 226, the wire housing 108 may be prevented from free axial movement in the axial direction 118. The potentially complementary nature of the clutch threads 221 with respect to the wire housing threads 109 may allow for rotation of the wire housing 108 about the central axis 204 to translate into gradual movement of wire housing 108 in the axial direction 118. Specifically, the gradual movement of the wire housing 108 may be determined by the pitch of the clutch threads 221 and the wire housing threads 109, and the rate of rotation of wire housing 108 about the central axis 204.

The clutch threads 221 and the wire housing threads 109 may have any suitable geometry. For example, the clutch threads 221 and the wire housing threads 109 may be square threads. This may strengthen the engagement between the clutch threads 221 and the wire housing threads 109. Additionally, it may enable the clutch 120 and the wire housing 108 to be made of lighter and/or softer materials (e.g. polymeric materials rather than metal, softer metals like aluminum rather than stainless steel).

Referring to FIG. 2D, the handle 102 may include a handle recess 224. The clutch 120 may be structured so that it is nested within the handle recess 224. The handle recess 224 may be formed of a part of the exterior of the handle 102. Among other forms, the handle recess 224 may be a groove, a recess, a hole, or a depression. The handle recess 224 may be semicircular in shape, or some other shape. The surface area of handle recess 224 may be larger than that of the outer surface of the clutch 120. Additionally, the handle recess 224 may also be sized and shaped to ergonomically fit the tip of a user's finger or thumb. For example, the handle recess 224 may allow a user of the handle 102 to comfortably engage and disengage the clutch 120. The handle recess 224 may also function as a reference point for a user when rotating the handle 102.

As shown in FIGS. 2C and 2D, the wire housing 108 may also include one or more wire housing threads 109, disposed on at least part of the outer surface of wire housing 108. The wire housing threads 109 may run for a portion of or the entire length of the wire housing 108. The wire housing threads 109 may control the movement of the wire housing 108. The wire housing threads 109, may have a thread pitch that complements the thread pitch of the clutch threads 221. For example, when the clutch 120 is in the engaged position 226 the clutch threads may engage with the wire housing threads 109. Rotation of the wire housing 108 (and thus the wire housing threads 109) about the central axis 204 may utilize the wire housing threads 109 to translate the rotation of into to incremental of the wire housing 108 in the axial direction 118.

The wire housing 108 may also include a wire aperture 230 extending therethrough. The wire aperture 230 may extend for the entire length of the wire housing 108. The wire aperture 230 may thus be accessible via either end of the wire housing 108 and may allow a guidewire to pass completely through the wire housing 108. Furthermore, the wire aperture 230 may be centered about the central axis 204.

The wire aperture 230 may be shaped so that it is symmetrical about the central axis 204. Alternatively, the wire aperture 230 may be shaped so that is asymmetrical about the central axis 204. The wire aperture 230 may be sized and shaped to at least partially contain a guidewire and may be wider or narrower at some points along its length. For example, the wire aperture 230 may be wider through the length of a first portion 232 and narrower along the length of a second portion 234 and a third portion 236. In having a greater width along the length of the first portion 232, the wire aperture 230 may accommodate the circumference of an end cap of a guidewire, while a lesser width along the length of a second portion 234 a third portion 236 may accommodate the body of a guidewire.

The wire aperture 230 may include a lock 238. The lock 238 may secure a guidewire within the wire aperture 230 and restrict the movement of a guidewire with respect to the wire housing 108. The lock 238 may thus translate motion (i.e. axial movement and/or rotation) of the wire housing 108 directly to a guidewire and allow a user to control the positioning of a guidewire with respect to the handle 102. Additionally, the lock 238 may be a button or some other actuator.

The lock 238 may be held in place by one or more lock springs 240, an aperture spring 242, or some other biasing mechanism or mechanisms. For example, the lock spring 240 and the aperture spring 242 may be coil springs. The lock spring 240 may effectively spring load the lock 238 so that it must be actively depressed by a user to toggle or otherwise move between a locked and an unlocked position (or vice versa). Additionally, the aperture spring 242 may bias or press against a surface created by the difference in width in the wire aperture 230 between the first portion 232 and the second portion 234. Thus, the aperture spring 242 may effectively restrict the movement of the lock 238 in the axial direction along some length of the wire aperture 230.

FIGS. 2A, 2B, and 2D show the driver housing 250 of the guidewire-capturing instrument 100. The driver housing 250 may detachably connect to a driver and translate motion of the handle 102 in any direction or rotation of the handle 102 about the central axis 204 to a driver (e.g. screwdriver bit 104), and thus to any component coupled to a driver. For example, the driver housing 250 may couple rotation of the handle 102 about the central axis to a driver (e.g. screwdriver bit 104) connected to it.

The driver housing 250 may include a driver aperture 252 extending in an axial direction 118 therethrough. The driver aperture 252 may extend for the entire length of the driver housing 250. Furthermore, the driver aperture 252 may be centered about a central axis 204. The driver aperture 252 may be shaped so that it is symmetrical about the central axis 204. Alternatively, the driver aperture 252 may be shaped so that is asymmetrical about the central axis 204. For example, the driver aperture 252 may be wider or narrower at some points along the direction of the central axis 204. The driver aperture 252 may be sized and shaped to encompass part of the wire housing 108. For example, the driver aperture 252 may circumferentially surround part of the third portion 236 of the wire housing 108. Part of the driver aperture 252 may also be sized and shaped to receive part of a driver.

The driver housing 250 may be encompassed at least partially by the handle 102 and contained at least partially by the central aperture 202. A portion of the driver housing 250 may threadedly engage with a nut 260. The nut 260 may mate or otherwise secure the driver housing 250 to the handle and 102 and prevent any movement of the driver housing 250 with respect to the handle 102. For example, the nut 260 may bias or press against a surface created by a difference in width in portions of the central aperture 202.

The nut 260 may also sandwich a washer 262 between a surface of the nut 260 and a surface created by a difference in width in portions of the central aperture 202. The washer 262 may distribute the load (i.e., the load that is created by the threaded engagement between the driver housing 250 and the nut) on the contacted portion of the handle 102. The washer 262 may also prevent damage to the handle 102 that may be caused by tightening the threaded engagement between the driver housing 250 and the nut 260.

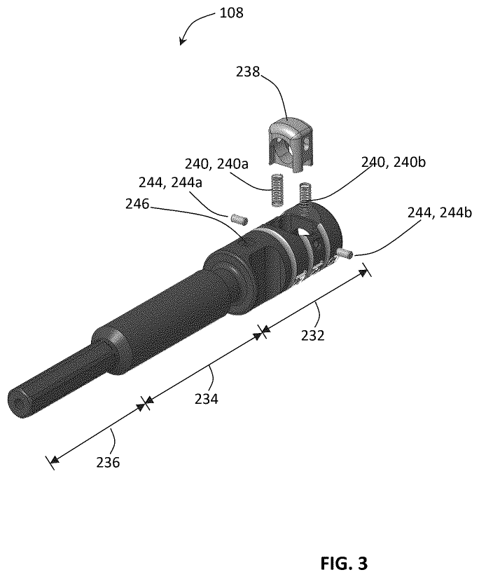

FIG. 3 is an exploded perspective view of an example of the wire housing of FIG. 2A. FIG. 3 includes the wire housing 108, the first portion 232, the second portion 234, the third portion 236, and the lock 238 shown in FIGS. 2C-2D. FIG. 3 also includes lock springs 240, 240a and 240, 240b, lock pins 244, 244a and 244, 244b, and a screw hole 246.

The wire housing 108 is shown in an enlarged simplified drawing that includes a first portion 232, a second portion 234, and a third portion 236. In this example, the wire housing 108 is shown without threads. However, in some examples, the wire housing 108 may have threads on any portion of its exterior surface, such as that which extends for the length of the second portion 234. Additionally, a portion of the wire housing 108 may have a polygonal shape or a generally cylindrical shape with flats. A flattened outer surface may reduce friction as the wire housing 108 rotates about the central axis 204 with respect to the handle 102. For example, the exterior surface of the wire housing 108 may have flats on it for a length that extends the length of the third portion 236. Accordingly, friction may be reduced as any part of the wire housing 108 (i.e. the third portion 236) rotates within the driver aperture 252 shown in FIG. 2D.

The wire housing 108 may include a lock 238 and in some embodiments, a first lock spring 240a and a second lock spring 240b (collectively referred to as lock springs 240). The first lock spring 240a and the second lock spring 240b may effectively spring load the lock 238 so that it must be actively depressed by a user to toggle between a locked and an unlocked position (or vice versa). Furthermore, the first lock spring 240a and the second lock spring 240b may be spaced with respect to the surface of the lock 238 to evenly bias the lock 238. For example, the first lock spring 240a and the second lock spring 240b may be positioned toward the ends of the bottom surface of the lock 238 to ensure relatively even locking force against the end cap or other portion of a guidewire positioned therein.

The wire housing 108 may also include one or more lock pins 244. For example, the wire housing 108 may include a first lock pin 244a and a second lock pin 244b. The first lock pin 244a and the second lock pin 244b may secure the lock 238 within the wire housing 108. For example, the first lock pin 244a and the second lock pin 244b may be sized and shaped to fit within slots or openings in the lock 238 and resist the biasing force of the first lock spring 240a and the second lock spring 240b, respectively. The first lock pin 244a and the second lock pin 244b may be removably placed so that removal thereof would allow for the biasing force of the first lock spring 240a and the second lock spring 240b to eject the lock 238 from the wire housing 108.

The wire housing 108 may also include one or more holes such as a screw hole 246, that may be sized and shaped to retain a fastener. For example, the screw hole 246 may have a first circumference that extends for a first length, partially into the wire housing 108, and a second, smaller, circumference that extends for a second length, further through the wire housing 108. Accordingly, this configuration would allow the screw hole to receive a screw, bolt, or fastener, such as the collar screw 210 shown in FIGS. 2B and 2C. The second, smaller circumference portion of the screw hole 246 may be threaded so that it may engage with a fastener such as the collar screw 210.

The screw hole 246 may receive and/or engage a fastener that interacts with a component other than the wire housing 108. For example, the fastener may interact with a collar 112, such as the collar 112 shown in FIG. 2A. In certain implementations, the screw hole 246 may be positioned along the surface of the first portion 232. The first circumference of the screw hole 246 may extend to a depth that allows for the head of a fastener (e.g. the collar screw 210) to protrude from the surface of the wire housing 108. Accordingly, the screw hole 246 may be sized and shaped such that, while inserted, a portion of a collar screw 210 protrudes from the wire housing 108 and abuts against the borders of a slot 211 in the collar 112. Thus, the slot 211 in the collar 112 and the position of the screw hole 246 with respect to the wire housing 108 may determine the bounds for which axial movement of the wire housing 108 with respect to the handle 102 may occur.

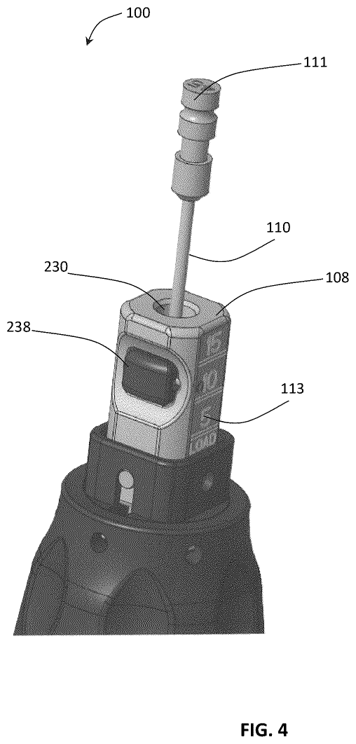

FIG. 4 is a perspective view of an example of the guidewire 110 and the wire housing 108 within the guidewire-capturing instrument 100 shown in FIGS. 1B-2D. FIG. 4 further includes a wire head 111, indicators 113, the wire aperture 230, and the lock 238.

FIG. 4 shows a portion of the guidewire-capturing instrument 100 and the wire housing 108 in an enlarged simplified drawing. The guidewire 110 may be inserted into the guidewire-capturing instrument 100 via the wire aperture 230. The guidewire 110 may include the wire head 111 positioned at one of the ends of the guidewire 110. The lock 238 may be positioned to receive and lock the wire head 111 of the guidewire 110.

The wire housing 108 may also include shapes, numbers, notches or other external markings such as indicators 113. The indicators 113 may be placed at predetermined intervals along the exterior surface of the wire housing 108. The indicators 113 may convey information to a user regarding the relative position various components of the guidewire-capturing instrument 100. For example, the indicators 113 may show the relative position of the wire housing 108, guidewire 110 or other components with respect to the handle 102.

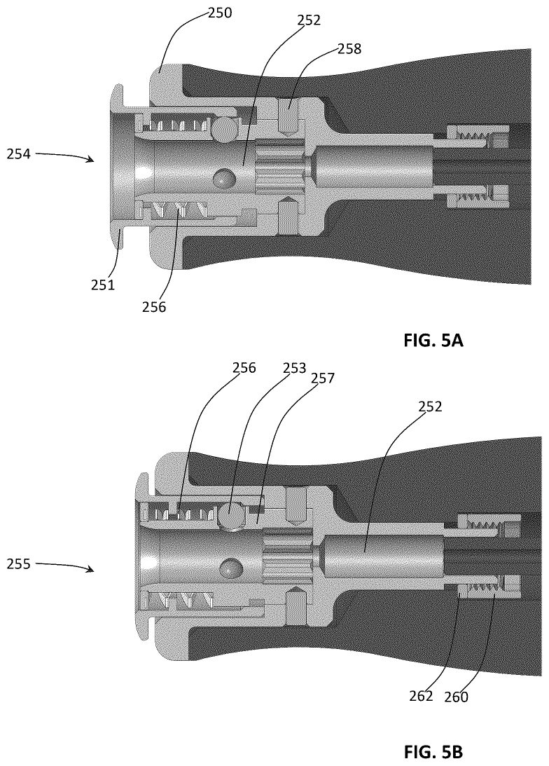

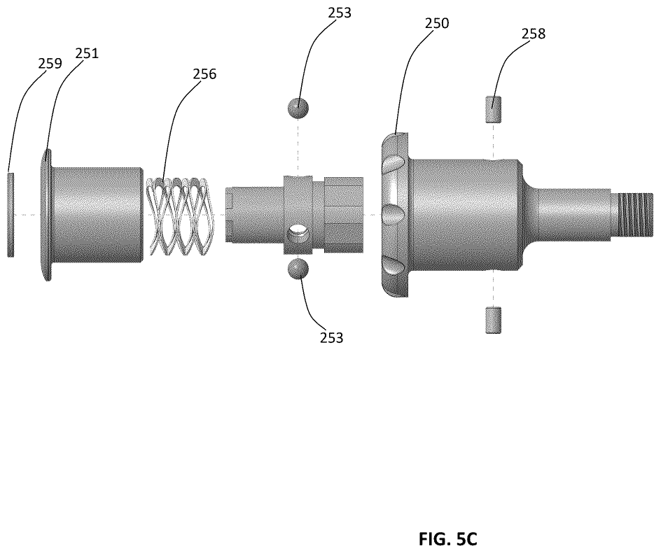

FIG. 5A is a cross-sectional view of an example of the driver housing 250 with the driver sleeve 251 in a first state 254. FIG. 5A includes the driver housing 250, the driver sleeve 251, and the driver aperture 252 of FIGS. 2B and 2D. FIG. 5A also includes a driver spring 256, and a driver pin 258.

FIG. 5B is a cross-sectional view of an example of the driver housing 250 with the driver sleeve 251 in a second state 255. FIG. 5B includes the driver aperture 252, and the driver spring 256 of FIG. 5A and the nut 260 and a washer 262 of FIG. 2D. FIG. 5B also includes a ball bearing 253, and a driver receiver 257.

FIG. 5C is an exploded side view of an example of the driver housing 251. FIG. 5C includes the driver housing 250, the driver sleeve 251, the ball bearings 253, the driver spring 256, and the driver pins 258 of FIGS. 5A-5B. FIG. 5C also includes the retention ring 259 of FIG. 2B.

Referring to FIGS. 5A-5C, the driver housing 250 is shown in more detail. The driver housing 250 may be configured to detachably mate, retain, and rotationally couple with connection features on various drivers. The driver housing 250 may include the driver sleeve 251 that interacts with additional features of the driver housing. For example, the driver sleeve 251 may be biased by a driver spring 256, or some other biasing mechanism. For example, the driver spring 256 may be a coil spring. The driver spring 256 may effectively spring load the driver sleeve 251 so that it can be actively depressed by a user to toggle or otherwise move between its settings.

For example, the driver spring 256 may bias or press against a protrusion on the driver sleeve 251 so that the default position of the driver sleeve 251 is in the first state 254. This may require a user to actively depress the driver sleeve 251 to move it into the second state 255, and when the user releases the driver sleeve 251, the driver spring 256 may urge the driver sleeve 251 back into the first state 254.

The driver sleeve 251 may interact with a locking mechanism to secure an object in place within the driver aperture 252. For example, the driver sleeve may interact with ball bearings 253 to secure at least a portion of a driver within the driver aperture 252. For example, the driver sleeve 251 may urge ball bearings from a first position to a second position. Accordingly, the ball bearings 253 may be positioned so that the first position may correspond with the first state 254, in which with the ball bearings 253 are being urged at least partially through a hole in a driver receiver 257. Furthermore, the ball bearings 253 may be positioned so that the second position may correspond with the second state 255, in which the ball bearings 253 are not being urged through a hole in a driver receiver 257 and a driver or any other item within the driver aperture 252 may have free axial movement or may be released from the driver aperture 252.

The driver housing 250 may also include a driver receiver 257. The driver receiver 257 may have the driver aperture 252 extending therethrough. The driver receiver 257 may be sized and shaped to receive and contain at least part of a driver and may have one or more holes through which one or more ball bearings 253 may protrude.

The ball bearings 253 may lock or release at least a portion of a driver. The ball bearings 253 may mate with a groove in an existing driver. Alternatively, freeing and blocking the ball bearings 253 may toggle or otherwise move the driver housing 250 between the first state 254 and the second state 255. For example, the first state 254 may correspond with the ball bearings 253 being urged into a position in which they protrude at least partially into the driver aperture 252 and settle into grooves in a driver therein. The first state 254 may correspond with at least a portion of a driver being retained and locked within the driver aperture. The second state 255 may correspond with a driver being released from within the driver aperture. This allows the driver housing 250 to engage and retain a driver with a standard connection, including but not limited to drivers for use in fluro-navigated or robotic surgical procedures.

In some embodiments, the driver housing 250 may include the retention ring 259 to retain a portion of the driver sleeve 251. The retention ring 259 may be configured to fit within at least a portion the driver sleeve 251. Additionally, the retention ring 259 may be welded to the outer surface of the driver aperture 252. Then retention ring 259 may rest against an internal part of the driver sleeve 251 and resist the biasing of the driver sleeve 251 by the driver spring 256. The retention ring 259 may prevent the driver sleeve 251 from dissociating from the driver housing 250. For example, the retention ring 259 may provide a backstop for the driver sleeve 251 and prevent the driver sleeve 251 from being fully ejected from a position within the driver housing 250.

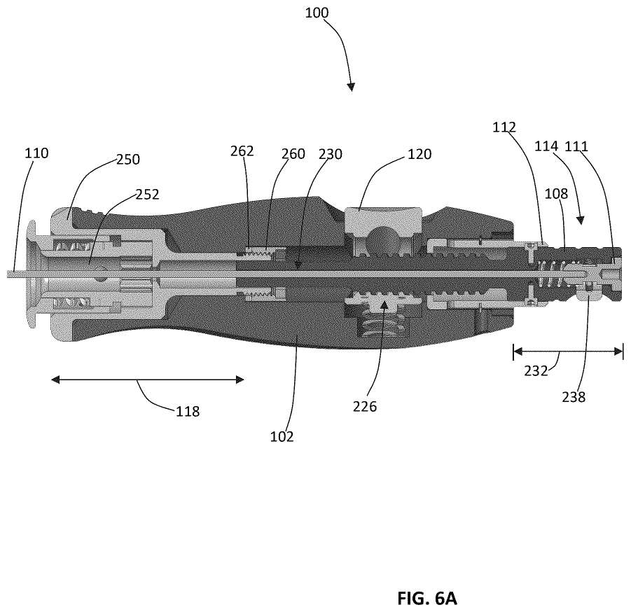

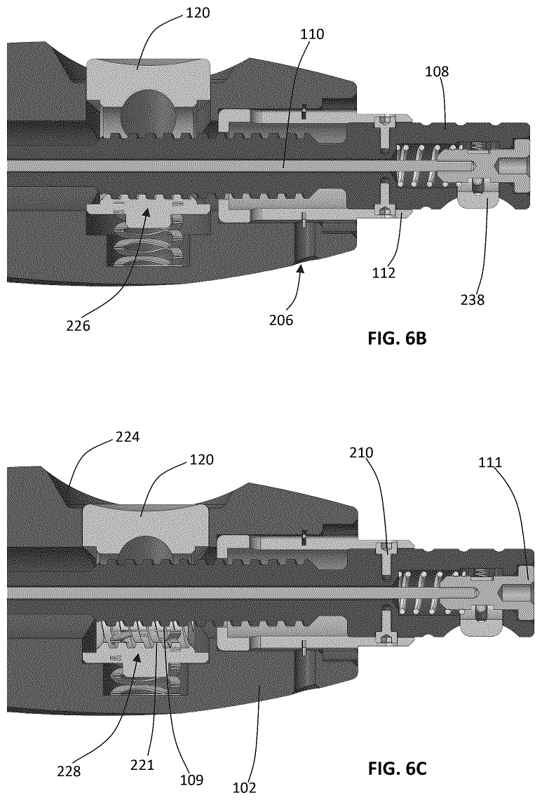

FIG. 6A is a cross-sectional view of an example of the guidewire-capturing instrument 100 with the guidewire 110 inserted. FIG. 6A includes the guidewire 110, and the wire head 111 of FIG. 4, shown in the first position 114 of FIG. 1A. FIG. 6A also includes the handle 102, the wire housing 108, the collar 112, the axial direction 118, the clutch 120, the wire aperture 230, the first portion 232, the lock 238, the driver housing 250, the driver aperture 252, the nut 260, the washer 262, of FIGS. 2C-2D, and further shows the clutch 120 in an engaged position 226.

FIG. 6B is a cross-sectional view of an example of the wire housing 108 with the guidewire 110 inserted with the clutch 120 engaged. FIG. 6B includes the wire housing 108, the guidewire 110, the collar 112, the access aperture 206, the lock 238, and the clutch 120 shown in the engaged position 226 of FIGS. 2C-2D and FIG. 6A.

FIG. 6C is a cross-sectional view of an example of the wire housing with a guidewire inserted with the clutch disengaged. FIG. 6C includes the handle 102, the wire threads 109, the wire head 111, the clutch 120, the collar screw 210, the clutch threads 221, and the handle recess 224, of FIGS. 2C-2D and FIG. 6A, and further shows the clutch 120 in a disengaged position 228.

Referring to FIGS. 6A-6C A guidewire 110 may be inserted into the wire housing 108 and pass through the wire aperture 230. The wire head 111 of the guidewire 110 may be locked in place by the lock 238 in the first portion 232 of the wire housing 108. The wire housing 108, and thus the guidewire 110 may be controlled by a user while the clutch 120 is in the engaged position 226. Alternatively, the wire housing 108, and thus the guidewire 110 may by controlled by a user while the clutch 120 is in the disengaged position 228, as shown in FIG. 6C.

The length of the guidewire 110 may vary. The guidewire 110 may extend through the wire housing 108 and into the driver housing 250, via the wire aperture 230 and the driver aperture 252. The guidewire 110 may extend out of the driver housing 250 while the wire housing 108 is in the first position 114. Additionally, the guidewire 110 may extend out of the wire housing 108 while the wire housing is in the second position 116. The guidewire 110 may extend beyond the length of the guidewire-capturing instrument 100 in the axial direction 118 so that the guidewire 110 may be implanted into tissue.

Also referring to FIGS. 6A-6C, although not shown in the drawings, a screwdriver bit 104 may be detachably connected to the handle 102 via the driver housing 250. In this case, the guidewire 110 may extend out of the driver housing 250 and through the screwdriver bit 104.

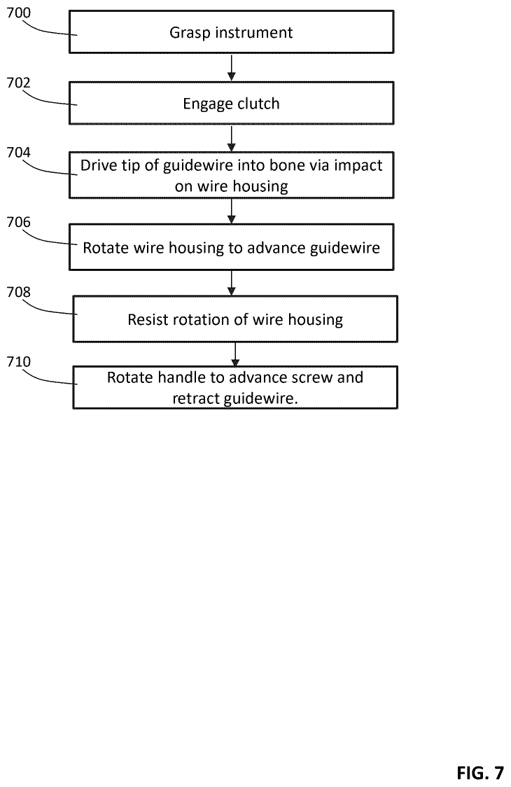

Referring to FIGS. 7-9, various methods of using the above-described device may be employed. A variety of workflows may allow a user (e.g. a surgeon) to tailor the process of guidewire insertion, screw implantation, and guidewire removal. Various methods may also give the user the ability to adapt to unforeseen events during the course of an operation. The following workflows may be examples of methods of use of a guidewire-capturing instrument 100 with a screwdriver bit 104 and a screw 106 connected and a guidewire 110 inserted therein.

FIG. 7 is a flow diagram of an example of a first method of use for the guidewire-capturing instrument with a driver, guidewire, and a screw. For example, a user may first grasp 700 the handle 102 of the guidewire-capturing instrument 100 with a guidewire 110 placed therein. Next the user may engage 702 the clutch 120, by placing the clutch 120 in the engaged position 226. The user may then drive 704 the tip of the guidewire 110 into the target tissue (i.e. bone or pedicle) via impact on the exposed end of the wire housing 108. The user may utilize an additional tool, such as a mallet to impact the end of the wire housing 108. When the user proceeds to drive 704 the wire housing 108, it may cause the guidewire 110 to move in the axial direction 118 and dock the tip of the guidewire 110 in the target tissue.

The user may then proceed to rotate 706 the wire housing 108 about the central axis 204, to advance the guidewire 110 in the axial direction 118 and further into the tissue. The user may utilize an additional tool to rotate 706 the wire housing 108, such as a T-handle. As the user proceeds to rotate 706 the wire housing 108, because the user previously acted to engage 702 the clutch 120, the wire housing 108 may gradually move in the axial direction 118. The axial movement of the wire housing 108 may be via the threaded engagement of one or more clutch threads 221 on the inside surface of the clutch 120 with the wire housing threads 109. Thus, the guidewire 110 may be gradually moved in the axial direction 118 through the guidewire-capturing instrument 100, the screwdriver bit 104, and the screw 106 and into the target tissue. The wire housing 108 and the guidewire 110 may move in the axial direction 118 while all the other components may not move in the axial direction 118.

Then, the user may resist 708 rotation of the wire housing by holding it in place, and preventing it from rotating about the central axis 204. The user may utilize an additional tool to resist 706 rotation such as a T-handle or another tool that may securely hold the wire housing 108. Then, while continuing to resist 708 rotation of the wire housing the user may rotate 710 the handle 102 about the central axis 204 to advance the screw 106 positioned at the end of the screwdriver bit 104 into the target tissue. Because of the threaded engagement created by the clutch 120, the rotation of the handle 102 in one direction about the central axis 204 may effectively result in relative rotation of the wire housing 108 in the opposite direction about the central axis 204 with respect to the handle 102. This rotation may effectively retract the guidewire 110 from the tissue while simultaneously advancing the screw 106 into the tissue.

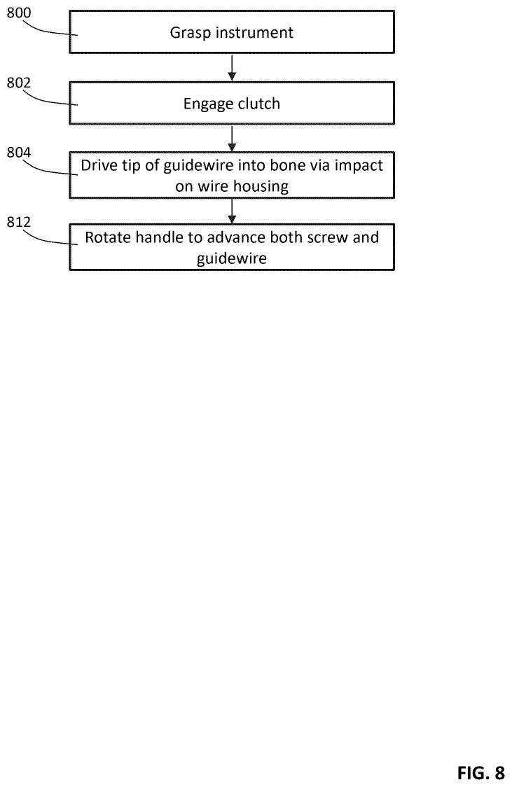

FIG. 8 is a flow diagram of an example of a second method of use for the guidewire-capturing instrument with a driver, guidewire, and a screw. Alternatively, after the user has proceeded to grasp 800 the guidewire-capturing instrument 100, engage 802 the clutch 120, and drive 804 the tip of the guidewire 110 into the tissue, the user may employ a different step. These steps where the user may grasp 800, engage 802, and drive 804 correspond to the steps where a user may grasp 700, engage 702, and drive 704 respectively, as shown in FIG. 7. The user may choose to rotate 712 the handle 102 about the central axis to advance both the screw 106 and the guidewire 110. This may effectively use the tip of the guidewire 110 as a tap for the screw 106 and advance both the screw 106 and the guidewire 110 in the axial direction and into the target tissue at the same time. After this step, it is possible that the guidewire 110 may be simply pulled out of the tissue via the channel created by the screw 106.

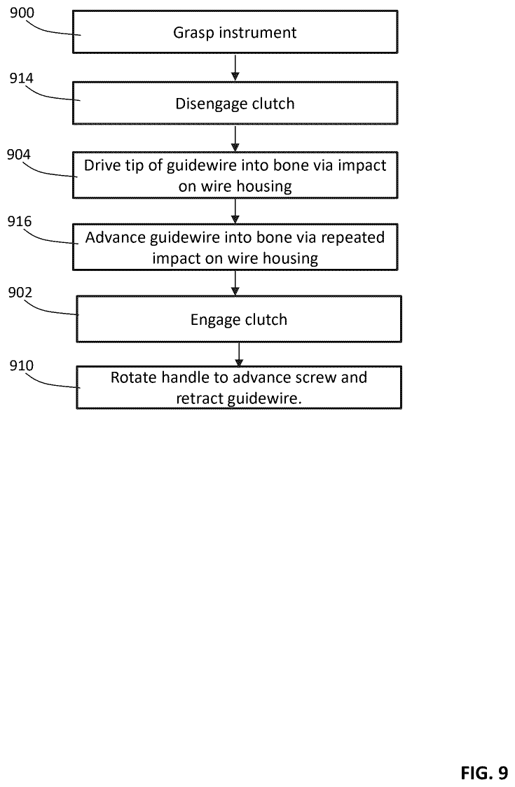

FIG. 9 is a flow diagram of an example of a third method of use for the guidewire-capturing instrument with a driver, guidewire, and a screw. Alternatively, the user may grasp 900 the guidewire-capturing instrument 100 and then may disengage 914 the clutch 120 by placing the clutch 120 in the disengaged position 228. Then, the user may drive 904 the tip of the guidewire 110 into the target tissue via impacting the end of the wire housing 108. Next, the user may advance 916 the guidewire 110 into the target tissue via repeated impact on the end of the wire housing 108. This may be facilitated by the fact that the clutch 120 may be in the disengaged position 228, which may correspond with free axial movement of the wire housing 108 in the axial direction 118. Next, the user may engage 902 the clutch 120 by placing the clutch 120 in the engaged position 226 and then rotate 910 the handle 102 about the central axis 204 to advance the screw and retract the guidewire. The steps where the user may grasp 900, drive 904, and engage 902 correspond to the steps where a user may grasp 700, engage 702, and drive 704 respectively, as shown in FIG. 7.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

Although several embodiments of the invention have been disclosed in the foregoing specification, it is understood that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. It is further envisioned that features from one embodiment may be combined or used with the features from a different embodiment described herein. Moreover, although specific terms are employed herein, as well as in the claims which follow, they are used only in a generic and descriptive sense, and not for the purposes of limiting the described invention, nor the claims which follow. Various features and advantages of the invention are set forth in the following claims.

What is claimed is:

1. A guidewire-capturing instrument, comprising:
a handle comprising a central aperture extending in an axial direction therethrough, wherein the central aperture is centered about a central axis extending in the axial direction;
a wire housing having a wire aperture extending in the axial direction therethrough, the wire aperture being sized and shaped to receive a guidewire;
the wire housing at least partially contained within the central aperture and selectively movable in the axial direction within the central aperture from a first position to a second position; and
a clutch connected to the handle and selectively movable between an engaged position and a disengaged position, the engaged position corresponding to a threaded engagement between the wire housing and the clutch, and the disengaged position corresponding to free axial movement of the wire housing within the central aperture between the first position and the second position.

2. The guidewire-capturing instrument of claim 1, further comprising:
a collar at least partially contained within the central aperture, the collar extending circumferentially around at least a portion of the wire housing, and
the collar being rotationally coupled to the wire housing such that rotation of the collar about the central axis corresponds with rotation of the wire housing about the central axis.

3. The guidewire-capturing instrument of claim 2, wherein the collar limits axial movement of the wire housing to define the first position and the second position.

4. The guidewire-capturing instrument of claim 3, wherein the collar comprises a slot extending in the axial direction with an abutment between the wire housing and a first end of the slot defining the first position and an abutment between the wire housing and a second end of the slot defining the second position.

5. The guidewire-capturing instrument of claim 2, wherein a surface of an end of the collar is sized and shaped to support engagement with a wrench.

6. The guidewire-capturing instrument of claim 1, further comprising:
a driver housing at least partially contained within the central aperture and having a driver aperture extending in the axial direction therethrough, the driver aperture being centered about the central axis and being sized and shaped to receive an end of a pedicle screwdriver, wherein the driver housing is rotationally coupled to the handle, and rotation of the handle about the central axis corresponds to rotation of the driver housing about the central axis; and
the driver aperture and the wire aperture combine to form a continuous open channel extending the length of the guidewire capturing instrument.

7. The guidewire-capturing instrument of claim 1, further comprising a lock incorporated into the wire housing and passing at least partially through the wire aperture, the lock positioned to selectively resist axial movement of the guidewire in the axial direction and rotation of the guidewire about the central axis.

8. The guidewire-capturing instrument of claim 5, wherein the wire aperture is sized and shaped to receive a k-wire.

9. A method of using a guidewire-capturing instrument, comprising:
grasping a handle of the guidewire-capturing instrument, the guidewire-capturing instrument comprising the handle, a wire housing, and a clutch, the handle having a central aperture extending in an axial direction therethrough, wherein the central aperture is centered about a central axis extending in the axial direction, the wire housing having a wire aperture extending in the axial direction therethrough, the wire aperture having a guidewire positioned therewithin, the wire housing at least partially contained within the central aperture and selectively movable in the axial direction within the central aperture from a first position to a second position, and the clutch connected to the handle and selectively movable between an engaged position and a disengaged position, the engaged position corresponding to a threaded engagement between the wire housing and the clutch, and the disengaged position corresponding to free axial movement of the wire housing within the central aperture between the first position and the second position; and
driving, while grasping the handle of the guidewire-capturing instrument, a tip of the guidewire into a pedicle via impact on a first end of the wire housing.

10. The method of claim 9 wherein the driving is performed while the clutch is in the engaged position.

11. The method of claim 10, further comprising rotating the handle about the central axis.

12. The method of claim 11, wherein the rotating the handle about the central axis is performed while resisting rotation of the wire housing about the central axis, thereby inducing relative rotation about the central axis between the handle and the wire housing.

13. The method of claim 12, wherein relative rotation of the handle about the central axis causes movement of the wire housing away from the first position and toward the second position.

14. The method of claim 12, wherein relative rotation of the handle about the central axis causes movement of the wire housing moving away from the second position toward the first position.

15. The method of claim 10, further comprising rotating the wire housing about the central axis while resisting rotation of the handle about the central axis.

16. The method of claim 15, wherein rotating the wire housing about the central axis causes movement of the wire housing away from the first position and toward the second position.

17. The method of claim 15, wherein rotating the wire housing about the central axis causes movement of the wire housing away from the second position and toward the first position.

18. The method of claim 9, wherein the driving is performed while the clutch is in the disengaged position.

19. The method of claim 18, wherein driving the tip of the guidewire into the pedicle comprises repeatedly impacting the first end of the wire housing and thereby moving the wire housing away from the first position and toward the second position.

20. The method of claim 19, further comprising:
moving, after the driving, moving the clutch to the engaged position; and
inducing, while the clutch is in the engaged position, relative rotation about the central axis between the handle and the wire housing to threadedly advance or threadedly retract the wire housing in the axial direction within the handle.
